(19) United States
(12) Patent Application Publication
(10) Pub. No.: US 2004/0006139 A1
(43) Pub. Date: Jan. 8, 2004

(54) Choline pyruvate, method for the production thereof, formulations containing choline pyruvate and the use thereof

(76) Inventors: Ralf Jager, Freising (DE); Martin Purpura, Bonn (DE); Gunter Ortenburger, Trostberg (DE); Ivo Pischel, Rossbach-Lache (DE)

Correspondence Address: Gary M. Nath, NATH & ASSOCIATES PLLC, 6th Floor, 1030 15th Street, N.W., Washington, DC, 20005, US

(21) Appl. No.: 10/465,897

(22) Filed: Jun. 20, 2003

Related U.S. Application Data
(63) Continuation of application No. PCT/EP01/13434, filed on Nov. 20, 2001.

(30) Foreign Application Priority Data
Dec. 22, 2000 (DE) 100 64 312.4

(51) Int. Cl. A61K 31/205; C07C 211/63

(52) U.S. Cl. 514/554; 562/577

(57) ABSTRACT

The invention relates to choline pyruvate of formula (I) wherein 0<n≦10, and is a stabile, physiologically compatible formulation containing said choline pyruvate, water and/or an organic solvent such as glycerine, or is present in a solid and preferably a powder form. The invention also relates to a method for producing choline pyruvate, whereby choline hydroxide is placed in a solvent, such as water or a methanol/water mixture, at a temperature of between 0 and 98° C., then pyruvric acid is added drop by drop in external cooling conditions and mixed until a pH value of between 5.0 and 8.0 is reached, the solvent is then removed and finally the obtained product is added to a physiologically compatible solvent. The choline pyruvate or the formulations containing choline pyruvate can be used preferably, as a food additive, as an animal food additive, as dietary substances, for improving general performances in the fields of sport, strength and endurance areas and, in deficiencies and illnesses of the central and/or peripheral nervous system and in the cosmetic field. Said choline pyruvate combines the positive physical and physiological properties of monosubstances such as choline and pyruvic acid and is particularly suitable for various applications in the medical field.

The subject matter of the present application is choline pyruvate, a method for the production thereof, a formulation containing the choline pyruvate and the use thereof.

Choline, i.e. hydroxyethyltrimethylammonium, was described as an animal component for the first time in 1862 by Strecker. Choline belongs to the group of B-vitamins and is synthesised endogenously from the essential amino acid methionine.

In addition to its free form, choline can be found in foods, for example, as a constituent of phospholipids. For example, bovine liver (3.5 oz) contains 532.28 mg of choline, eggs (large) contain 282.32 mg, nuts (1 oz) contain 27.91 mg, coffee (6 oz) contains 19.29 mg, 1 cup of breast milk contains 10.29 mg and one cup of baby food contains 11.0 mg of choline. The average daily intake is approximately 1 g, the minimum daily requirement is approximately 0.8 g.

In spite of the plurality of nutritional sources containing choline, a general reduction in the daily intake of choline is occurring in a protein free diet and as a result of the increasing cut-back in fatty foods, and it thus seems sensible to additionally supplement the diet with choline.

However, the bioavailability of the different sources of choline varies as does the corresponding release profile. Approximately 60% of the oral intake of choline is converted into di- or trimethylamine, namely by intestinal bacteria.

Choline chloride and phosphatidyl choline are rated as GRAS (generally recognised as safe, 21 CFR, Secs. 182, 184). The “lowest-observed-adverse-effect level” (LOAEL) of choline is 7.5 g/day, which results in an upper tolerable daily dose of 3.5 g, taking into consideration an element of uncertainty, i.e. approximately 3.5 times the normal average daily intake.

However, doses of up to 30 g per day (for a body weight of 70 kg) are used for the treatment of specific disorders of the nervous system.

The side effects observed at higher doses were nausea, perspiration, anorexia, an unpleasant body odour and cardiac irregularities.

Numerous positive effects on health have been proven for choline:

Choline plays an important role in the structural integrity of the cell membrane, as a methyl group donor in the intermediary metabolism, in neurotransmission, in transmembrane communication and in lipid-cholesterol transport and metabolism.

Choline is involved, for example as acetylcholine, in the transmission of nerve impulses, e.g. to the muscle fibres. Consequently, the plasma choline level decreases during intensive endurance activities. Therefore, in marathon runners for example, the average plasma choline level decreases during the competition by approximately 40% (L. A. Conlay et al., Decreased plasma choline concentrations in marathon runners, N. Eng. J. Med. 1986, 315 892). Just as clear decreases could be observed in swimmers (R. Coates et al., The effect of choline on fatigue, energy levels and performance in college swimmers. Presented at: Nutrition and Physical Activity to Optimize Performance and Well-being; Thursday, Spr. 6, 1995, Atlanta, Ga.) and triathletes (H. N. von Allworden et al., The influence of lecithin on plasma choline concentrations in triathletes and adolescent runners during exercise, Eur. J. Appl. Physiol. Occup. Physio. 1993, 67, 87).

The plasma choline level can be kept constant if choline is administered before intensive activity (B. W. Sandage et al., Choline compounds and performance in humans. Presented at: NIH workshop on the role of dietary supplements for physically active people, Monday, Jun. 3, 1996, Bethesda, Md.). Supplementation with choline leads to increased performance in specific sporting fields: thus, in a placebo-controlled study, a performance increase of approximately 3% could be observed in long-distance runners over a distance of 20 miles following the intake of 2.8 g of choline chloride (B. W. Sandage et al. Choline citrate may enhance athletic performance, Physiologist. 1992, 35, 236a). Increases in performance and a lower degree of fatigue could be observed in swimmers (R. Coates et al., The effect of choline on fatigue, energy levels and performance in college swimmers. Presented at: Nutrition and Physical Activity to Optimise Performance and Well-being; Thursday, Apr. 6, 1995, Atlanta, Ga.).

Choline also influences reproductive and development processes: it therefore plays a decisive role in the development of the brain and learning ability, it influences the folate status, improves spermatic movement and is part of the platelet activating factor which, for example, plays an important role in egg implantations. Choline is, however, also required for a normal functioning of the liver and is involved in the metabolism of homocysteine which can, as regards the function of the heart, increase the risk of CVD. Finally, the role which choline also plays in the brain cannot be ignored, for example regarding memory performance or Alzheimer's disease; choline also belongs to the few substances which surmount the blood/brain barrier and directly reach the brain cells. It is furthermore an important factor in diets which are low in fat and cholesterol and beneficially influences nerve activity (cholinergic neurones).

Pyruvic acid tends towards oligomerisation and polymerisation and must therefore be stabilised by the formation of suitable salts called pyruvates. Zinc pyruvate and calcium pyruvate are described in addition to sodium and potassium pyruvate, however, calcium pyruvate, is not stable as ≧2.5 hydrate and calcium pyruvate monohydrate (cf. DE-OS 197 29 786.2) is the sole stable form of calcium pyruvate. Liquid formulations of the known pyruvate salts are generally unstable.

It is sufficiently well known that the salts of pyruvic acid have valuable physiological, therapeutic and dietetic properties.

Pyruvates are widely used to increase endurance and strength in the field of sport, to reduce weight and body fat, as a protective substance for body cells and tissue, in particular for protecting cardiovascular, hepatic, nephrotic, peritoneal and neuronal tissues, as a substance for inhibiting radical formation and as a radical intercepting substance in body cells and tissues (also synovial tissue); pyruvates are furthermore now diversely used in the field of health, as a dietary supplement, as vulnerary agents and for the treatment of kidney diseases (e.g. acute renal failure or nephrolithiasis).

In addition to the known salts of pyruvic acid having inorganic cations, pyruvates having organic compounds, such as, for example, creatine pyruvate (DE-OS 196 53 225), have also been described more frequently in recent years.

Choline pyruvate has hitherto not yet been characterised as a substance. U.S. Pat. No. 5,321,050 and EP 656 382 both describe methods for producing modified polyisocyanurate foams, which revert to catalysts, the general formula of which also contains choline pyruvate. The material properties of choline pyruvate are, however, not specified in these two documents. Hydrates of choline pyruvate in particular are not mentioned.

The object of the present invention, i.e. to provide a chemically defined choline pyruvate, a method for the production thereof as well as formulations containing choline pyruvate, thus arose from the known positive properties of the individual compounds choline and pyruvate and due to the lack of characteristics of the compound.

This object is solved by means of a choline pyruvate having the following formula:

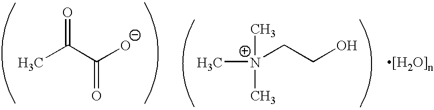

wherein 0<n≦10. n is preferably 1 or 2.

It was surprisingly found that the obtained compound is relatively easily accessible, has clear characteristics and also has good stability. It can furthermore be used in formulations without any problems.

Also claimed is a method for the production of choline pyruvate, in which:

a) an alkaline choline salt such as, for example, choline hydroxide or choline hydrogen carbonate, is placed in a solvent, preferably water or a 30 to 50% methanol/water mixture, at a temperature of between 0 and 98° C., in particular of between 10 and 45° C.,

and then, in two alternative steps, either

b₁) pyruvic acid is added drop by drop in external cooling conditions and whilst being stirred until a pH value of between 5.0 and 8.0, in particular between 6.5 and 7.5, is reached,

and/or

b₂) a pyruvate, preferably sodium, potassium, calcium, magnesium or zinc pyruvate, is added.

Subsequently, the invention allows in the following step

c) for the solvent to be removed, preferably under vacuum,

and finally, as required,

d) a physiologically compatible solvent is added to the obtained product.

A particularly preferred method is claimed, in which a choline hydroxide is used which is obtained by the reaction of suitable choline salts such as, for example, choline chloride, and a suitable base such as, for example, sodium hydroxide. The reaction of the choline salt with the base can also be carried out in situ.

According to the object, the present invention does not only claim the choline pyruvate itself and a method for the production thereof, but rather also a stable, physiologically compatible formulation which contains the choline pyruvate having the above formula.

The invention preferably comprises a corresponding formulation which contains water and/or an organic solvent, with it being particularly preferred that glycerine can be contained. However, in addition to these particularly suitable solvents of water and glycerine, all other suitable solvents which do not negatively influence the positive physical and physiological properties of the claimed choline pyruvate can be used.

In this regard, the physiologically compatible solvent used in step d) above is to be very broadly defined and, in addition to the already cited solvents of water and glycerine, can also include, for example, fruit juices, sugar solutions, infusion solutions, etc.

In addition to the described liquid formulations, the present invention also claims formulations which are in a solid state and, in particular, in a powdered state.

From the number of possible solid formulations, chewable tablets and effervescent tablets have proven to be successful as the form of administration of choline pyruvate.

The invention allows for the formulation to comprise the choline pyruvate in amounts of 1 to 99 wt-% and preferably in amounts of 10 to 70 wt-%, regardless of the physical state of the choline pyruvate formulations.

In order to take into account the wide range of areas claimed, the present invention includes, in particular, formulations which, in addition to their own content, i.e. choline pyruvate and, where applicable, the compatible solvent components, also contain correspondingly suitable formulation adjuvants and/or physiologically compatible or effective adjuncts.

The formulation adjuvants obviously have to be adapted to the specific use purposes, and above all methylcelluloses, SiO₂, stearates, dissolving agents, flavouring agents, preservatives and release agents come into question for this reason.

Sugar, alcohols, vitamins, trace elements, amino acids, colouring and flavouring agents and texturants are particularly suitable as physiologically compatible or effective adjuncts.

In addition to the choline pyruvate itself and formulations containing choline pyruvate, the present invention also claims the use of the choline pyruvate and the formulations containing choline pyruvate, preferably as a dietary supplement, as an animal food additive, as dietary substances, for improving general performances in sport and in the field of strength and endurance, in functional foods, for the prevention and, preferably supporting, treatment of reproduction disorders, for deficits and diseases of the central and/or peripheral nervous system, in particular for disturbances in memory performance and degenerative cerebral diseases, for the disturbed metabolism of the liver and/or the heart, for arteriosclerosis as well as for reducing weight. In this regard, the idea of the invention includes, in particular, the use of choline pyruvate for the production of a medicament for diseases of the central nervous system, preferably in the form of an injectable solution.

The effect of the choline pyruvate as a radical interceptor and/or as an antioxidant and above all as a dietary supplement or animal food additive must be particularly mentioned.

However, the present invention also includes the use of the choline pyruvate in the field of cosmetics. The choline pyruvate can be used in cosmetic preparations to clean the skin and hair, to improve foam and to condition the hair as well as to achieve a pleasant feeling of the skin. Owing to its nature, the compound according to the invention is often used with surfactants and other additives to influence surface properties, with all the cited formulations being able to contain the known additives such as, for example, surface-active agents, surfactants or emulsifiers and thickening agents.

With the claimed choline pyruvate, the present invention provides a new compound which combines the positive physiological properties of both choline and pyruvate, with the compound itself being produced in a very easy manner and leading to stable formulations which can be used in a wide field of application.

The following examples clarify these positive aspects of the present invention.

### EXAMPLES

### Example 1

### Method of Production (Best Embodiment)

12.5 g (47 mmol) of choline hydroxide (45% in methanol) and water are used at 25° C., and then, whilst being stirred, 4.3 g (49 mmol) of pyruvic acid are added drop by drop under an exothermic reaction (increase in temperature of up to 45° C.) and in external cooling conditions until the solution has a largely neutral pH value of 6.5 to 7.0. The solvent was subsequently removed by means of a waterjet vacuum using a 40° C. warm water bath. The product obtained was a clear, yellow liquid which remained stable during 12 hours of storage in a refrigerator. 10.1 g of product were obtained in this manner, said product smelling faintly of amine, having a pH value of 7.0 and a density d of 1.13. The water content of the obtained product was 8.4 wt-% (according to the Karl-Fischer method). This corresponds to n equals 1.

Elemental analysis: C: 46.27% (calculated 45.92%), H: 9.27% (calculated 9.15%), N: 6.63% (calculated 6.70%).

IR (KBr): ν=3398 (s), 1708 (s), 1620 (s)

1H-NMR (D₂O, 300 MHz): 4.00 (m, 2H, HO—CH₂—), 3.49 (m, 2H, —CH₂—N—), 3.15 (s, 9H, N(CH₃)₃), 2.31 (s, 3H, H₃C—CO—).

13C-NMR (D₂O, 75.5 MHz): 205.0 (CO—COO), 170.3 (COO), 67.4 (HO—CH₂), 55.6 (CH₂—N), 53.9 (N(CH₃)₃), 26.5 (CO—CH₃).

### Example 2

### Stability Test Using the Product Obtained in Example 1

| | | Day 4 | Day 21 | Day 42 | Day 103 |
|---|---|---|---|---|---|
| HPLC content | Pyruvate | 35.3% | 35.8% | 35.5% | 35.9% |
| | Parapyruvate | 2.7% | 2.6% | 2.6% | 2.8% |
| IC content | Choline | 50.6% | 50.6% | 50.7% | 50.6% |
| Appearance | | clear | clear | clear | clear |

### Example 3

### Stability Test of a 20% Aqueous Choline Pyruvate Solution

2.0 g of choline pyruvate were dissolved in 8 g of water and the 20% clear and light yellow solution obtained in this manner, having a pH value of between 5 and 6 and a density d of 1.05, was stored at room temperature.

| | | Day 4 | Day 21 | Day 42 | Day 103 |
|---|---|---|---|---|---|
| HPLC content | Pyruvate | 7.9% | 7.7% | 7.8% | 7.8% |
| | Parapyruvate | 0.7% | 0.8% | 0.6% | 0.5% |
| IC content | Choline | 10.6% | 10.7% | 10.6% | 10.6% |
| Appearance | | clear | clear | clear | clear |

### Example 4

### Use

4.1) A 29 year old female (K.C.) reported a significant performance increase of 4% in a run over marathon distance (26.2 miles) following a daily supplement of 10 g of choline pyruvate.

4.2) A clear and lasting weight reduction of approximately 3% was observed in a 57 year old male (H.K.) following a daily supplement of 5 g of choline pyruvate, as well as an increase in the fat-free body mass linked with an increase in his overall well-being.

1. Choline pyruvate having the formula 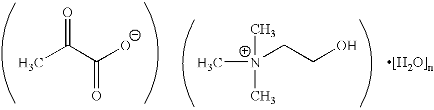
wherein 0<n≦10.

2. Method for producing choline pyruvate according to claim 1, characterised in that
a) an alkaline choline salt such as, for example, choline hydroxide or choline hydrogen carbonate, is placed in a solvent, preferably water or a 30 to 50% methanol/water mixture, at a temperature of between 0 and 98° C., in particular of between 10 and 45° C., then
b₁) pyruvic acid is added drop by drop in external cooling conditions and whilst being stirred until a pH value of between 5.0 and 8.0, in particular between 6.5 and 7.5, is reached
and/or
b₂) a pyruvate, preferably Na, K, Ca, Mg or Zn pyruvate, is added,
subsequently
c) the solvent is removed, preferably under vacuum,
and, as required,
d) a physiologically compatible solvent is added to the obtained product.

3. Method of production according to claim 2, characterised in that a choline hydroxide is used which is obtained by the reaction of suitable choline salts such as, for example, choline chloride, and a suitable base such as, for example, sodium hydroxide.

4. A stable, physiologically compatible formulation containing choline pyruvate according to claim 1.

5. A formulation according to claim 4, characterised in that said formulation contains water and/or an organic solvent, preferably glycerine.

6. A formulation according to claim 4, characterised in that said formulation is given in a solid, preferably powdered, state.

7. A formulation according to one of claims 4 to 6, characterised in that said formulation contains choline pyruvate in amounts of 1 to 99 wt-% and preferably in amounts of 10 to 70 wt-%.

8. A formulation according to one of claims 4 to 7, characterised in that said formulation contains formulation adjuvants and/or further physiologically compatible adjuncts.

9. Use of choline pyruvate according to one of the previous claims as a dietary supplement, as an animal food additive, as dietary substances, for improving general performances in sport and in the field of strength and endurance, in functional foods, for the prevention and, preferably supporting, treatment of reproduction disorders, for deficits and diseases of the central and/or peripheral nervous system, in particular for disturbances in memory performance and for degenerative cerebral diseases, for the disturbed metabolism of the liver and/or the heart, for arteriosclerosis and for weight reduction, as well as in the field of cosmetics.

10. Use according to claim 9 for the production of a medicament for diseases of the central nervous system, preferably in the form of an injectable solution.
